(19) United States
(12) Patent Application Publication
(10) Pub. No.: US 2005/0136419 A1
(43) Pub. Date: Jun. 23, 2005

(54) Method and apparatus for nanogap device and array

(75) Inventor: Luke P. Lee, Orinda, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/814,609

(22) Filed: Mar. 29, 2004

Related U.S. Application Data
(60) Provisional application No. 60/458,885, filed on Mar. 28, 2003.

(51) Int. Cl. C12Q 1/68; C12M 1/34; H01L 21/00

(52) U.S. Cl. 435/6; 435/287.2; 438/1

(57) ABSTRACT

A method and/or system using a nanogap capacitor device and methods for construction of the same.

### RELATED APPLICATIONS

This application claims priority from provisional patent application 60/458,885 entitled METHOD AND APPARATUS FOR NANOGAP DEVICE AND ARRAY by Luke Lee, filed on 28 Mar. 2003. The entire contents of that filing are hereby incorporated herein by reference.

### FIELD OF THE INVENTION

The present invention relates to methods and/or system and/or apparatus involving nanogap structures, including fabrication and methods that can be involved in use of such structures. In specific embodiments, the invention involves methods and/or system and/or apparatus involving nanogap structures for detecting substances of interest. In other embodiments, the invention involves methods and/or system and/or apparatus involving nanogap structures useful in other applications, such as for information storage.

### BACKGROUND OF THE INVENTION

The discussion of any work, publications, sales, or activity anywhere in this submission, including in any documents submitted with this application, shall not be taken as an admission that any such work constitutes prior art. The discussion of any activity, work, or publication herein is not an admission that such activity, work, or publication existed or was known in any particular jurisdiction.

Various strategies have been explored in microfabrication. Microfabricated devices in silicon and other materials have found uses in electronic circuits and some detection and diagnostic devices as well as in micromachine devices. Furthermore, various strategies have been proposed for detecting substances and/or molecules and/or chemicals and/or compounds of interest. These strategies have been proposed for a number of applications such as, but not limited to, biologic assays and/or diagnostic tests, tests for drugs, explosives and/or other contraband substances, tests in manufacturing processes for desired or undesired substances, tests in food or manufacturing processes for contamination and/or pollution constituents, etc.

Some strategies have been discussed that utilize fabricated devices as part of a detecting device and/or system. Discussion of various of such strategies and related technology can be found in references cited in this submission. Furthermore, diagnosis is an essential tool in the health care industry. The role of diagnosis is expanding, particularly within the context of screening and prevention. Diagnosis in many instances involves detecting molecules of interest.

Biosensor technologies have been widely studied. They have been based on electrochemical (Palecek et al., 1998; Marrazza et al., 1999), optical (Piunno et al., 1994), mass sensitive (Okahata et al., 1998), and acoustic wave transducers (Zhang et al., 1998).

The dielectric properties of molecules depend on electron transfer, atomic bonds, and the large-scale molecular structure. The characteristic time scales range from 10⁻¹² s for electrons, 10⁻⁹ s for atomic bonds, to 10⁻³ s for molecular structures. Therefore, when an oscillatory field perturbs molecules, they respond differently depending on the frequency. The low frequency response indicates the large-scale molecular structure changes like the conformation changes of DNA during hybridization from single stranded DNA to double stranded DNA or conformation changes that occur due to polypeptide (e.g., protein) folding.

The dielectric response of DNA in solution has been widely studied (Saif et al., 1991; Mandel, 1977; van der Touw and Mandel, 1974; Takashima, 1963, 1966, 1967; Baker-Jarvis et al., 1998). Dielectric relaxation of DNA solution occurs at least at three different frequency regions: α (a few kilohertz or lower), β (roughly from 1 MHz to 1 GHz), and γ (above 1 GHz). Among them, α relaxation has a large dielectric increment which is dependent on the length of the DNA molecule. And it reflects the migration of counter ions over the entire dimension of the DNA molecule. This close relationship between α relaxation and the electrical double layer (EDL) makes it difficult to do dielectric measurements at low frequency. Early dielectric spectroscopy of DNA solution was limited at low frequency due to the noise from EDL impedance, which dominates the measured capacitance.

The EDL impedance is due to the accumulation of counter ions near the electrode surface, which becomes charged when in contact with an electrolyte (Hunter, 1993). The distribution of electrolyte ions in the vicinity of a charged surface was first predicted by Gouy (Gouy, 1910) and Chapman (Chapman, 1913) by assuming the Boltzmann distribution for ions and ion interaction with a mean potential governed by Poisson equation, i.e. Poisson-Boltzmann (PB) equation. The basic feature is the build up of an electrical double layer adjacent to the electrode surface.

### SUMMARY

The present invention, in specific embodiments, involves novels methods for using and/or manufacturing a nanogap device and other devices having a small gap and/or feature size. Such devices may be useful in detection devices as herein described an in other microfabricated systems or devices, such as electronic circuits, hybrid devices and microfabricated machines.

The present invention, in specific embodiments, provides a device for detecting substances of interest, particular biological sequence molecules, such as DNA, RNA, amino acids, polypeptides, etc., and provides advantages in cost and manufacturability over other proposed devices.

In further embodiments, the invention involves assays utilizing methods and devices as described herein. In specific example embodiments, an array of nanogap devices with attached molecular probes can be used as a detection array for detecting biologic molecules and other molecules of interest without the need for labeling or florescence detection.

In further embodiments, the invention involves a nanogap capacitor-based biosensor designed to detect DNA hybridization and/or other ligand binding events. For example, such a sensor can measure the capacitance difference in dielectric properties between single-stranded DNA and double-stranded DNA to detect hybridization without DNA labeling or can measure other ligand binding reactions. Applications include BioMEMS, Lab on a chip, etc. Features and benefits according to specific embodiments of the present invention are that capacitance measurements provide fast and sensitive in-situ monitoring of hybridization/binding without labeling.

In further embodiments, the invention involves a nanogap capacitor-based biosensor designed to detect molecular conformational changes in a sample. Such conformational changes can indicate, for example, whether a sample contains a protein that has folded. Using real-time detecting of changes, the invention can determine through an easily performed electrical detection whether a sample contains polypeptides that have folded under applied conditions.

Electrical and magnetic modulation can be employed to improve the sensitivity of sensors according to specific embodiments of the present invention as described in this submission.

While example detectors according to specific embodiments of the present invention is described herein as used for performing a biological assay, it will be understood to those of skill in the art that a detector according to specific embodiments of the present invention can be used in a variety of applications for detecting substances of interests. These applications include, but are not limited to: detecting pollutants in effluent from a manufacturing facility; detecting contaminants in foodstuffs; detecting the presence of a desired substance (such as petroleum components) in a mining or exploration operation; insuring the presence of desired elements in a manufacturing output, etc. Devices according to specific embodiments of the present invention can also be used for non-detecting applications, such as information storage using molecular reactions in a hybrid circuit.

Thus, from the teachings provided herein, it will be understood that according to specific embodiments, the present invention provides one or more of (1) novel fabrication methods for nanogap dielectric junctions; (2) label-free molecular (e.g., DNA, RNA, other sequences) sensor comprising nanogap junctions and sensing or probe molecules; (3) methods for improving sensitivity of nanogap dielectric measurements by increasing the overall effective areas; optimizing the boundary conditions of different biopolymers; or investing dielectric relaxations & amplifications; (4) computational models for the interfacial polarization dynamics of DNA/RNA and other molecules of interest in nanogap junctions & nanofluidic devices; (5) nanofluidic controls can be incorporated in nanogap junction arrays to improve functionality; and (6) sensor and/or storage chips integrating array of nanogap junctions with nanofluidic controls that provide further enhanced functionality.

Other Features & Benefits

The invention and various specific aspects and embodiments will be better understood with reference to drawings and detailed descriptions provided in this submission. For purposes of clarity, this discussion refers to devices, methods, and concepts in terms of specific examples. However, the invention and aspects thereof may have applications to a variety of types of devices and systems. It is therefore intended that the invention not be limited except as provided in the attached claims and equivalents.

Furthermore, it is well known in the art that systems and methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

In some of the drawings and detailed descriptions below, the present invention is described in terms of the important independent embodiment of a biologic assay and/or array system and components thereof. This should not be taken to limit the invention, which, using the teachings provided herein, can be applied to a number of other situations.

All references, publications, patents, and patent applications cited in this submission are hereby incorporated by reference in their entirety for all purposes.

References

Baker-Jarvis, J., Jones, C. A., and Riddle, B., 1998. Electrical Properties and Dielectric Relaxation of DNA in Solution. NIST technical note, 1509.
Basu, S. and Sharma, M. M., 1994. Effect of dielectric saturation on disjoining pressure in thin-films of aqueous-electrolytes. J. Colloid Interface Sci. 165, 355-366.
Berggren et al Electroanalysis, vol.11, p156, 1999.
Berggren, C.; Bjarnason, B.; Johansson, G. “Capacitive Biosensors.” Electroanalysis, (2001) Vol. 13, No. 3, p.173-180.
Bernengo and M. Hanss, “Four electrode, very low frequency impedence comparator for ionic solutions,” Rev. Sci. Instrum. vol. 47, pp. 505-508, 1976.
Berney, H. et al Sensors and Actuators B vol. 68, p. 100, 2000.
Biesheuvel, P. M., 2001. Implications of the charge regulation model for the interaction of hydrophilic surfaces in water. Langmuir, 17, 3553-3556.
Biesheuvel, P. M., 2001. Simplifications of the Poisson-Boltzmann equation for the electrostatic interaction of close hydrophilic surfaces in water. J. Colloid Interface Sci., 238, 362-370.
Chapman, D. L., 1913. A Contribution to the Theory of Electrocapillarity. Philos. Mag., 25, 475-481.
Fricke and Curtis, J. Phys. Chem., vol. 41, 729, 1937.
Gouy, G., 1910. Sur la constitution de la charge électrique à la surface d'un electrolyte. J. Phys. Radium, 9, 457-468.
Hunter, Foundation of Colloid Science, p. 337, Oxford science publications, 1987.
Hunter, R J, 1993. Introduction to Modem Colloid Science, Oxford University Press Inc., New York.
Mandel, M., 1977. Dielectric properties of charged linear macromolecules with particular reference to DNA. Ann. NY Acad. Sci. 303, 74-87.
Marrazza, G., Chianella, I., Mascini, M., 1999. Disposable DNA electrochemical sensor for hybridization detection. Biosensors and Bioelectronics 14, 43-51.
Okahata, Y., Kawase, M., Niikura, K., Ohtake, F., Furusawa, H., Ebara, Y., 1998. Kinetic measurements of DNA hybridisation on an oligonucleotide-immobilised 27-MHz quartz crystal microbalance. Analytical Chemistry 10, 1288-1296.
Palecek, E., Fojta, M., Tomschik, M., Wang, J., 1998. Electrochemical biosensors for DNA hybridization and DNA damage. Biosensors and Bioelectronics 13, 621-628.
Piunno, P. A. E., Krull, U. J., Hudson, R. H. E., Damha, M. J., Cohen, H., 1994. Fiber optic biosensor for fluorometric detection of DNA hybridization. Analytica Chimica Acta 288, 205-214.
Saif, B., Mohr, R. K., Montrose, C. J., and Litovitz, T. A., 1991. On the mechanism of dielectric-relaxation in aqueous DNA solutions. Biopolymers 31, 1171-1180.
Schwan, “Alternating current electrode polarization,” Biophysik, vol. 3, pp. 181-201,1966.
Schwan, Electrical properties of tissue and cell suspensions, 147-209, Academic press, 1957.
Sebaek, O., Lee, J. S., Jeong, K. H., and Lee, L. P., 2003. Minimization of electrode polarization effect by nanogap electrodes for biosensor applications. Proc. 16th IEEE International Micro Electro Mechanical Systems 2003, 52.
Sidhom, G.; Imam, M. “Evaluation of Serum Laminin as a Tumor Marker in Breast Cancer.” Int. J. Clin. Lab. Res. (1999) Vol. 29, p. 26-29
Takashima, Electrical properties of biopolymers and membranes, Adam Hilger, 1989.
Takashima, S., 1963. Dielectric dispersion of DNA. J. Mol. Biol. 6, 455-467.
Takashima, S., 1966. Dielectric dispersion of deoxyribonucleic acid .2. J. Phys. Chem. 70, 1372.
Takashima, S., 1967. Effect of ions on dielectric relaxation of DNA. Biopolymers 5, 899.
van der Touw, F., and Mandel, M., 1974. Dielectric increment and dielectric-dispersion of solutions containing simple charged linear macromolecules .1. theory. Biophys. Chem. 2, 218-230.
Zhang, H., Tan, H., Wang, R., Wei, W., Yao, S., 1998. Immobilisation of DNA on silver surface of bulk acoustic wave sensor and its application to the study of UV-C damage. Analytica Chimica Acta 374, 31-38.
Zhmud, B. V., Meurk A., and Bergström L., 1998. Evaluation of surface ionization parameters from AFM data. J. Colloid Interface Sci. 207, 332-343.

### BRIEF DESCRIPTION OF THE DRAWINGS

FIG. 1 illustrates an example nanogap junction and example nanogap junction array device according to specific embodiments of the present invention.

FIG. 2 illustrates an example experimental set-up uses for measuring nanogap capacitance according to specific embodiments of the invention.

FIG. 3 illustrates an example of measurements of the electric potential between two electrodes for various channel widths (with a concentration of solution is 0.1 mM of 1:1 electrolyte) according to specific embodiments of the invention.

FIG. 4 illustrates an example measurements of the capacitance of a buffer solution with different concentrations measured according to specific embodiments of the invention.

FIG. 5 illustrates an example measurements of the capacitance electric potential between two electrodes for various nanogap channel widths according to specific embodiments of the invention.

FIG. 6 illustrates an example measurements of the capacitance variation with immobilization and hybridization at different frequencies using heteronucleotides.

FIG. 7 illustrates attached receptor molecules (e.g., single stranded DNA) and attached bonded receptor molecules (e.g., double stranded DNA) in a nanogap junction device to illustrate a model of operation according to specific embodiments of the present invention.

FIGS. 8A and B illustrate example immobilization techniques according to specific embodiments of the present invention.

FIG. 9 illustrates an example of capacitance data for laminin detection using an example nanogap device and a SAM-based immobilization protocol according to specific embodiments of the present invention.

FIG. 10 illustrates an alternative example of capacitance data for laminin detection using an example nanogap device according to specific embodiments of the present invention.

FIG. 11 illustrates different conformations of Cytochrome c a well-known monomeric protein.

FIG. 12 is a graph showing frequency versus dielectric measurement of water as different pH values used as a control.

FIG. 13 is a graph showing frequency versus dielectric measurement of a solution of Cytochrome c at 1 μM and a different pH values.

FIG. 14 is a more detailed view of the graph shown in FIG. 13.

FIG. 15 illustrates an example method for performing a diagnostic test according to specific embodiments of the present invention.

FIG. 16 illustrates an example method for constructing a nanogap device according to specific embodiments of the present invention.

FIG. 17A-B are SEM images of an example nanogap device according to specific embodiments of the present invention.

FIG. 18 illustrates an alternative fabrication process flow diagram for creating planar nanogap spaced electrodes according to specific embodiments of the present invention.

FIG. 19 is a block diagram illustrating a six step fabrication process for fabricating a nanogap device using gold (Au) deposition and to form gold electrodes with a nanogap channel according to specific embodiments of the invention.

FIG. 20 is a block diagram illustrating alternative fabrication steps of the method shown in FIG. 19 according to specific embodiments of the invention.

FIG. 21 is a block diagram illustrating a gold electrode nanogap junction with detector molecules arranged therein according to specific embodiments of the invention.

FIG. 22 is a block diagram illustrating a top view showing four first electrodes to the left, each forming a nanogap junction with an electrode to the right according to specific embodiments of the invention.

FIG. 23 is a block diagram illustrating a top view showing ten first electrodes, each forming a nanogap junction with a central gold reference electrode according to specific embodiments of the invention.

FIG. 24 is a block diagram illustrating a floating or planar nano-gap capacitor with a transparent (e.g., quartz) substrate providing an optical window according to specific embodiments of the invention.

FIG. 25 is a block diagram showing steps in an example fabrication process for fabricating a transparent planar nanogap capacitor according to specific embodiments of the invention.

FIG. 26 shows SEM images of an example fabricated floating nanogap capacitor with 50 nm nanogap height between the bottom and upper N⁺ poly-Si upper electrodes.

FIG. 27 is a block diagram showing a representative example logic device in which various aspects of the present invention may be embodied.

FIG. 28 (Table 1) illustrates an example of diseases, conditions, or statuses for which at least one gene is differentially expressed that can evaluated according to specific embodiments of the present invention.

FIG. 29 illustrates a scanning electron micrograph (SEM) image of nanogap junction features of an example nanogap device according to specific embodiments of the present invention.

### DESCRIPTION OF SPECIFIC EMBODIMENTS

### 1. Definitions

The following definitions may be used to assist in understanding this submission. These terms, as well as terms as understood in the art should be used as a guide in understanding descriptions provided herein.

The term “microarray” or “high-density array” refers to a substrate or collection of substrates or surfaces bearing a plurality of array elements (e.g. discrete regions having particular moieties, e.g. proteins, nucleic acids, etc., affixed thereto), where the array elements are typically present at a density of greater than about 10 elements/cm², preferably greater than about 100 elements/cm², more preferably greater than about 1000 elements/cm², and most preferably greater than about 10,000 elements/cm², or 100,000 elements/cm².

The term “microarray substrate” refers to a substrate suitable for the formation of a microarray comprising a plurality of array elements. The microarray substrate need not be used as a component of a microarray.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide. Preferred “peptides”, “polypeptides”, and “proteins” are chains of amino acids whose α carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to peptide mimetics such as amino acids joined by an ether as opposed to an amide bond.

The terms “binding partner” or “binding moiety”, or a member of a “binding pair”, or “cognate ligand” refers to molecules that specifically bind other molecules to form a binding complex such as antibody/antigen, lectin/carbohydrate, nucleic acid/nucleic acid, receptor/receptor ligand (e.g. IL-4 receptor and IL-4), avidin/biotin, etc.

The term ligand is used to refer to a molecule that specifically binds to another molecule. Commonly a ligand is a soluble molecule, e.g. a hormone or cytokine, that binds to a receptor. The decision as to which member of a binding pair is the ligand and which the “receptor” is often a little arbitrary when the broader sense of receptor is used (e.g., where there is no implication of transduction of signal). In these cases, typically the smaller of the two members of the binding pair is called the ligand. Thus, in a lectin-sugar interaction, the sugar would be the ligand (even if it is attached to a much larger molecule, recognition is of the saccharide).

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The term “specifically binds”, as used herein, when referring to a biomolecule (e.g., protein, nucleic acid, antibody, etc.), refers to a binding reaction which is determinative of the presence of a biomolecule in a heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all, to other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C., sometimes 20° or 25° lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 37° C. using standard hybridization solutions, e.g., containing (e.g. 50%) formamide (see, e.g., Sambrook (1989) Molecular Cloning: A Laboratory Manual (2nd ed.) Vol. 1-3, Cold Spring Harbor Laboratory, Cold Spring Harbor Press, New York, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “target analyte” refers to an agent that is to be detected and/or quantified. The agent can be virtually any biologic and/or chemical compound. Target analytes include, but are not limited to nucleic acids (e.g. certain mRNAs), proteins (e.g. certain immunogens), sugars, carbohydrates (e.g. bacterial carbohydrates, growth factors, transcription factors, hormones, cytokines, and the like.

The phrase “adsorbed to a surface” refers to the attachment of a moiety (e.g., a molecule) to a surface produced by drying a solution comprising that moiety on the surface. The attachment can be by one or more of a variety of mechanisms including, but not limited to covalent bonds, ionic interactions, hydrophobic interactions, van der Waals interactions, and the like.

A “substrate” is a, preferably solid, material suitable for the attachment of one or more molecules. Substrates can be formed of materials including, but not limited to glass, plastic, silicon, germanium, minerals (e.g. quartz), semiconducting materials (e.g. doped silicon, doped germanium, etc.), ceramics, metals, etc.

“Transparent” as used herein indicates a material able to effectively pass an electromagnetic radiation of interest that can be used to measure or view or image some quality through the material.

### 2. Overview

In specific embodiments, the invention involves a novel bio-sensor element, methods, and/or array. Some earlier bio-sensor array solutions require fluorescent labeling, which is time consuming and can require complex optical apparatus.

In specific embodiments, the invention involves novel methods for measuring and/or determining characteristics of oligonucleotide sequences, such as DNA or RNA, using dielectric measurements in nanogap junctions. While the dielectric properties of DNA solution have been widely investigated, early approaches are limited at low frequency by the parasitic noise due to the electrical double layer (EDL) impedance. Electrodes separated by Nanogap junctions (referred to at times herein as nanogap electrodes) according to specific embodiments of the invention can serve effectively as biomolecular junctions because the gap size (e.g., on the order of 5-100 nm) minimizes electrode polarization effects regardless of frequency.

The effects of the EDL interaction between two parallel nanogap electrodes can be modeled by solving the Poisson-Boltzmann equation for equilibrium state. When the gap size is smaller than the EDL thickness, the dependence of the nanogap capacitance on the ionic strength is insignificant, which is important in using the capacitance change as an indicator of target molecule existence or conformation.

### 3. Example Array Overview

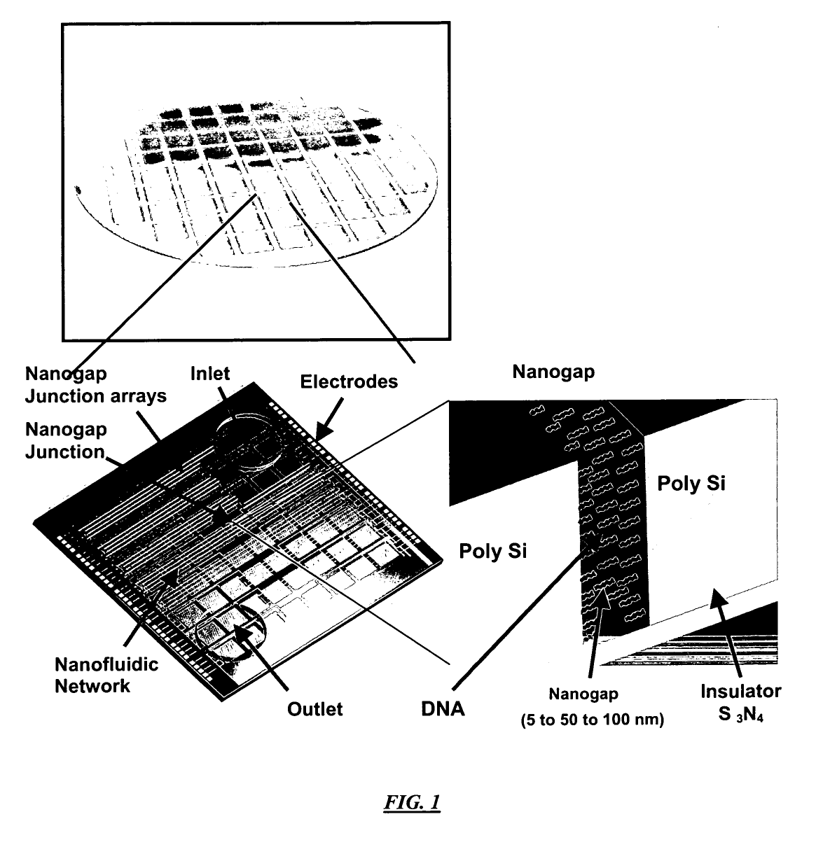

To provide a context for understand specific embodiments of the present invention, consider FIG. 1. FIG. 1 illustrates an example nanogap junction and example nanogap junction array device according to specific embodiments of the present invention. In this example, an 8×8 array of 64 nanogap capacitors is shown. The array is addressable using standard electronic addressing and a cover and inlet and outlet for fluidic sample introduction is shown. The example nanogap junction shown is a vertical nanogap having polySi electrodes, though other nanogap constructions are possible, including the specific device configurations discussed herein. In this example, DNA strands are shown attached to the electrodes of the nanogap which can act as specific hybridization detectors as further described herein. Alternatively, a system such as that shown can be used with RNA, polypeptides, antibodies, or other molecules suitable for a nanogap dielectric analysis as further described herein.

### 4. Nanogap Device Operational Overview

Electrical Properties of Nanogap Junction and Minimization Of Electrode Polarization Effect

Nanogap-electrode junctions according to specific embodiments of the present invention provide improved detection by dielectric effects by reducing the effects of electrode polarization using one or more techniques as described herein. Previous work has indicated that electrode polarization can cause errors in determining impedance of biological samples in solution. The unwanted electrical double layer (EDL) impedance (i.e. electrode polarization impedance) is believed to be caused by the accumulation of ions on the electrode. The accumulation of the ions on the electrode makes it difficult to determine the electric property of the sample. This effect becomes more dominant when the measurements are done in the low frequency (e.g., < about 1 kHz) and high conductive solutions.

Using nanogap electrode-based biomolecular measurements with a two-electrode setup according to specific embodiments of the invention minimizes electrode polarization effects using double layers that overlap and therefore reduce potential drops inside of the electrode gap. According to specific embodiments of the invention, nanoscale (e.g., < about 100 nm, down in some cases to 10 nm to several Angstroms in some embodiments) electrode spacing can be used to reduce these unwanted effects.

To isolate this polarization impedance effect, previous work analyzed the system and represented it with an equivalent two-electrode circuit model. Several methods have been proposed to minimize this polarization impedance effect, such as by measuring the electric properties with different electrode spacing. It is, however, assumed that induced parasitic capacities are the same for different spacing of the electrodes, which is not true when the polarization effect is large. A different method proposed for avoiding the electrode polarization is to coat the electrode with platinum black, which is cumbersome. Four electrode measurement has also been suggested as the best way to avoid the polarization effect, but this requires a relatively complex measurement setup.

Several equivalent circuits have been proposed to clarify the contributions from biological impedance and electrode polarization impedance. Serial connection between the electrode polarization impedance and biological impedance is reasonable in relatively large electrode spacing since double layer is confined only to very thin layer near. The observed capacitance (C_{measured}) and resistance (R_{measured}) are given by

$${C}_{measured} = {C}_{sample} + \frac{1}{{ω}^{2} {R}^{2} {C}_{double layer}} \qquad ( 1 )$$

R_{measured}=Rₛₐₘₚₗₑ+R_{doublelayer}+ω²R²C²Rₛₐₘₚₗₑ (2)

When measuring in low frequency, equation (1) shows that the electrode polarization term will severely obscure the observed capacitance values due to the second term of right side. The resistance values will be also distorted by the electrode polarization since double layer resistance (R_{double layer}) will go up as the frequency decreases. Therefore, the electrode polarization effect makes it extremely difficult to measure the actual impedance of the biological materials in a conventional setup.

Nanogap Electrode

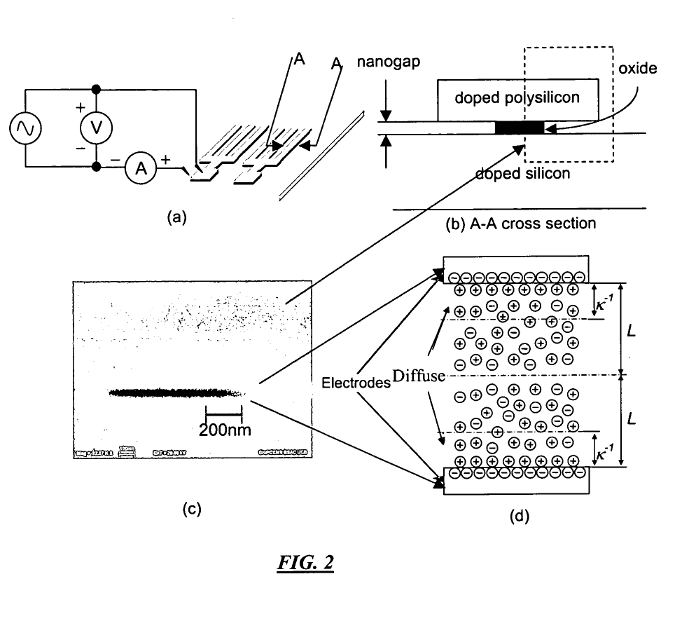

According to specific embodiments of the invention, using two electrodes with a nanogap junction measurement can minimize the electrode polarization effects regardless of frequency. FIG. 2 illustrates an example experimental set-up uses for measuring nanogap capacitance according to specific embodiments of the invention. FIG. 2(a) is a block diagram of a two-wire impedance measurement setup; FIG. 2(b) is a block diagram of the electrodes of the nanogap; FIG. 2(c) illustrates an SEM of a nanogap fabricated using same masks for the tested nanogap; FIG. 2(d) is a block diagram of a nanogap filled with electrolytes (Two length scales, gap size L and electrical double layer thickness κ⁻¹, exist).

For nanogap measurement system, the double layer prevails across the whole space between the electrodes rather than being confined to near the electrodes. Therefore the parallel circuit model of double layer impedance and sample impedance is more appropriate to describe the situation.

The total impedance and observed capacitance and resistance are represented by the following equations.

$$\begin{matrix}{C}_{measured} = \frac{{C}_{double layer}}{1 + {ω}^{2} {C}_{double layer}^{2} {R}_{double layer}^{2}} + {C}_{sample} & ( 3 ) \\ \frac{1}{{R}_{measured}} = \frac{{ω}^{2} {C}_{double layer}^{2} {R}_{double layer}}{1 + {ω}^{2} {C}_{double layer}^{2} {R}_{double layer}^{2}} + \frac{1}{{R}_{sample}} & ( 4 )\end{matrix}$$

From the above equations can be observed the clear difference between the parallel connection and series connection. When the frequency goes to zero, C˜C_{double layer}+Cₛₐₘₚₗₑ and R˜Rₛₐₘₚₗₑ. Therefore, in low frequency regions, the observed resistance values are almost equal to the sample resistance values. And for the capacitance, it has the parasitic term which is equal to double layer capacitance. In large gap size, where series equivalent circuit model is applied, the parasite double layer effects are amplified in low frequency regions even if the double layer capacitance value is small. In electrodes with nanogap spacing according to specific embodiments of the invention, the parasite double layer effects will not be amplified in low frequency regions and only contribute by its own values.

Capacitance Measures

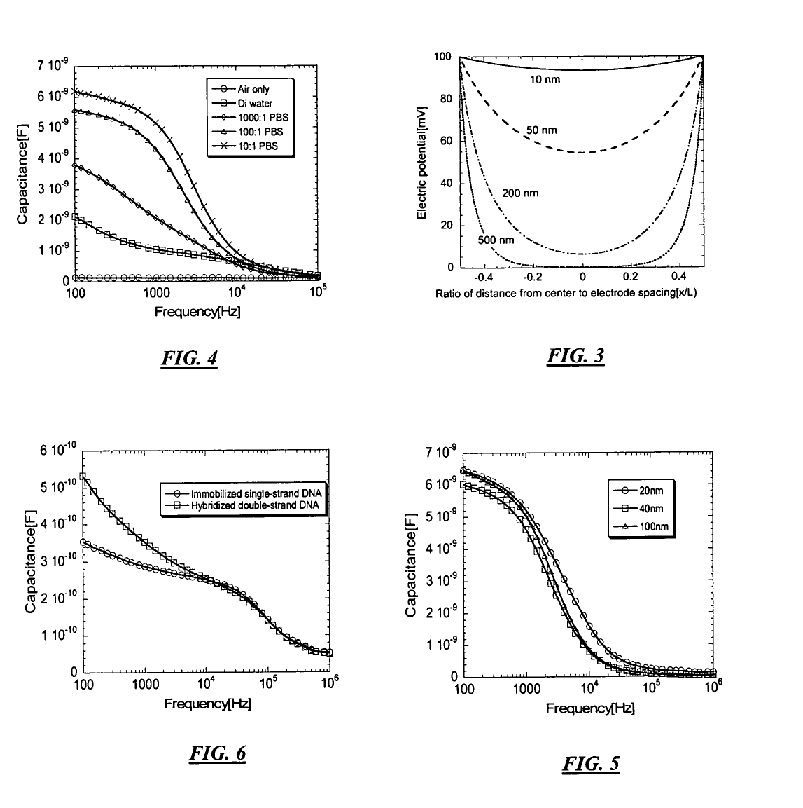

FIG. 3 illustrates an example of measurements of the electric potential between two electrodes for various channel widths (with a concentration of solution is 0.1 mM of 1:1 electrolyte) according to specific embodiments of the invention. As shown in the figure, as the gap sizes reduces down from about 100 nm to about 10 nm, the potential drop per unit thickness is larger and larger, and therefore the capacitance is larger. At small dimensions, the double layer overlaps, which reduces the potential drop and the capacitance. These electric potential distributions may be modeled by the Poisson-Boltzmann equation. The almost flat profile of electric potential in at around the 10 nm gap indicates that the net ion densities are zero in most regions even around the electrode so that double layer capacitance due to electrode polarization can be reduced dramatically.

In order to demonstrate the effects of the double layer, the capacitance of buffer solutions with various concentrations and gap sizes was measured and it was observed that the capacitance increases as the buffer solution concentration increases. This trend can be explained by the fact that double layer thickness will be smaller with higher concentration. Most of electric potential drop will happen in the thinner layer, so the resulting capacitance is higher. This result indicates that in some embodiments lower concentration of buffer solution may provide more sensitive sensors. FIG. 4 illustrates an example measurements of the capacitance of a buffer solution with different concentrations measured according to specific embodiments of the invention. Di water denotes deionized water. PBS is Phosphate-Buffered Saline and 1000:1 PBS denotes the solution which is diluted 1000 times by adding the deionized water.

FIG. 5 illustrates an example measurements of the capacitance electric potential between two electrodes for various nanogap channel widths according to specific embodiments of the invention In this example, the concentration of solution is 0.1 mM of 1:1 electrolyte and the example nanogap junctions according to specific embodiments of the invention measured are 20 nm, 40 nm and 100 nm.

### 5. Example Dielectric Detections

Using any of the nanogap structures described herein, a variety of different molecular detections can be performed based on dielectrical measurements. An example of several of these detections is provided below followed by further discussion of fabrication of nanogap devices and different device configurations.

Hybridization

To provide a further example according to specific embodiments of the invention, the dielectric measurement of DNA solution was performed using nanogap electrode arrays according to the invention. It has previously been described that a single-stranded (ss) DNA shows different dielectric behavior from a double-stranded (ds) DNA, especially in low frequency region. Therefore, in one application, the invention measures a dielectric property (e.g., a capacitance) between nanogap junction electrodes to detect DNA hybridization and therefore, in particular embodiments, the sequence of the DNA. A nanogap electrode junction fabricated according to specific embodiments of the invention has proven effective to dielectric properties of DNA solutions before and after hybridization.

The changes of dielectric properties before and after hybridization steps at different frequencies using different hetero-nucleotides are shown in FIG. 6. The experimental procedures are as follows. First oligonucleotides (sequence: TGCAGTTTTCCAGCAATGAG) were immobilized on the SAM coated nanogap electrodes for 3 hrs at room temperature and were washed with Phosphate-Buffered Saline (PBS) solution, and then dielectric properties of the immobilized DNA coated device were measured. For hybridization, matched probes (sequence: ACGTCAAAAGGTCGTTACTC) were added for 3 hrs at room temperature, washed with PBS solution. Finally, changes of dielectric properties due to hybridization were measured. In dielectric measurements, the capacitance and dissipation factor of DNA included solution were investigated at frequency range of 100 Hz to 1 MHz, 0V_{DC} bias and 25 mV_{AC} signals using a (HP4284A) LCR meter. In this example, the maximum changes (40-50%) of capacitance are observed at 100 Hz after hybridization, which is much more sensitive the previous detection measurements (2˜3% signal change)

According to further embodiments of the present invention, the minimization of electrode polarization effect in a nanogap junction can be verified by a theoretical model and the mechanism of dielectric increments after the hybridization can be explained by the counterion polarization theory. A representative formula is as follows:

$$\vert \frac{Δ {ɛ}_{dsDNA}}{{Δɛ}_{ssDNA}} \vert \approx \frac{{a}^{2}}{bR} \frac{{( 1 + p / 2 )}^{2}}{2 {( 1 + p )}^{2}} \frac{( 1 + {ω}^{2} {τ}_{ssDNA} )}{( 1 + {ω}^{2} {τ}_{dsDNA} )} .$$

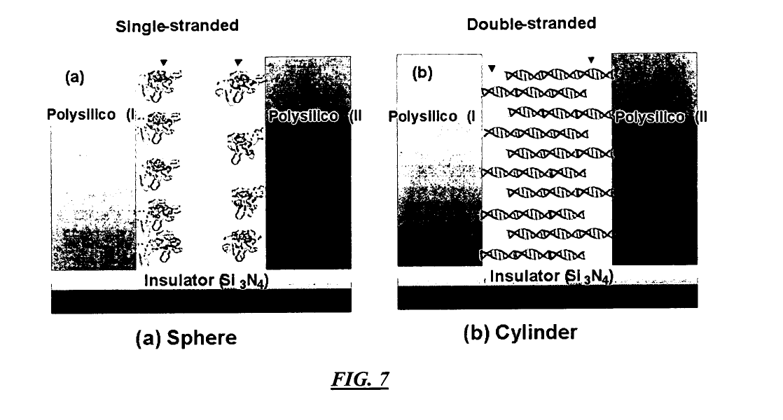

FIG. 7 illustrates attached receptor molecules (e.g., single stranded DNA) and attached bonded receptor molecules (e.g., double stranded DNA) in a nanogap junction device to illustrate a model of operation according to specific embodiments of the present invention.

Detecting Moiety/Anti-Body Binding

According to further embodiments, the invention can be used to detect a variety of receptor/binding reactions, such as binding of a specific antibodies to its antigen.

As an example and for experimental verification, methods and devices according to specific embodiments of the present invention were been used for the specific dielectric sensing of laminin bound to immobilized anti-laminin using a 60 nanometer electrode gap size. Observed capacitance changes at 100 Hz on the selective binding of laminin over a human serum albumin (HSA) control is 25% for a laminin concentration of 50 μg/ml; a larger change than believed to be previously reported because of low frequency measurements. It is believed that this occurs because of the reduction of electrode polarization effects from overlapping electrical double layers between the two nanogap electrodes and the increased fractional volume occupied by the sensed proteins. As an example of a diagnostic assay using the present invention, the concentration of the P1 fragment of laminin present in blood serum is indicative of cancerous growth and can be measured with a laminin-based immunosensor.

Immobilization

In some applications, a nanogap sensor according to specific embodiments of the invention may be used in conjunction with an attached (or immobilized) molecule. As examples, two methods of anti-laminin (mouse) immobilization on electrode surfaces are discussed here. However, any method of molecular immobilization can be used with specific devices according to the invention.

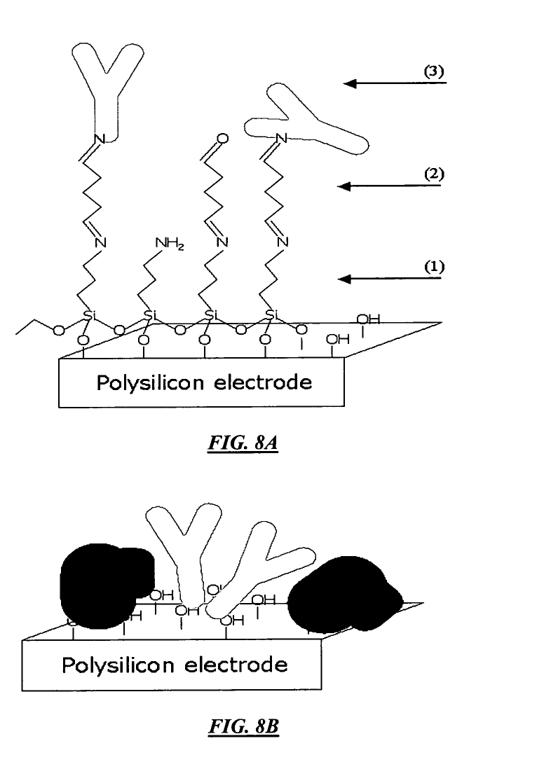

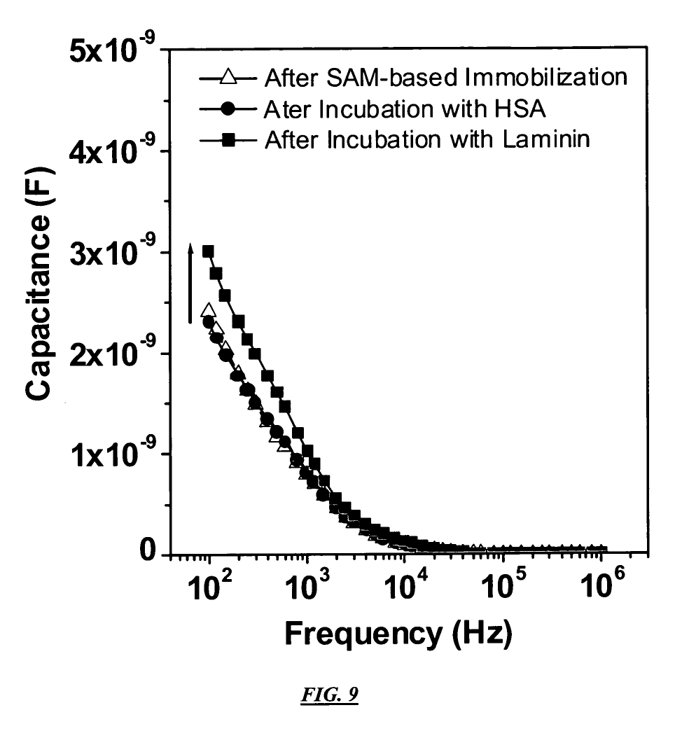

In one example method, (FIG. 8A) a self-assembled monolayer of (3-aminopropyl)triethoxysilane (APTS) is immobilized to the surface using standard techniques. A glutaraldehyde linker is then covalently attached to the primary amine of the immobilized APTS. The antibody is then covalently linked by various amines on lysine and arginine residues to the free aldehyde. The remaining reactive aldehydes are then quenched by immobilizing triethanolamine.

In the second procedure (FIG. 8B), the antibody is adsorbed directly to the electrode surface and bovine serum albumin (BSA) is also incubated on the surface to block other adsorption sites.

FIGS. 8A and B illustrate example immobilization techniques according to specific embodiments of the present invention. These figures are functional illustrates and not drawn to scale.

For both immobilization methods dielectric measurements of anti-laminin—laminin were investigated over a frequency range of 100 Hz to 1 MHz, with 0V DC bias and 25 mV AC signals using a (HP4284A) LCR meter. Measurements were taken after immobilization steps to determine the buildup of the biosensing layer.

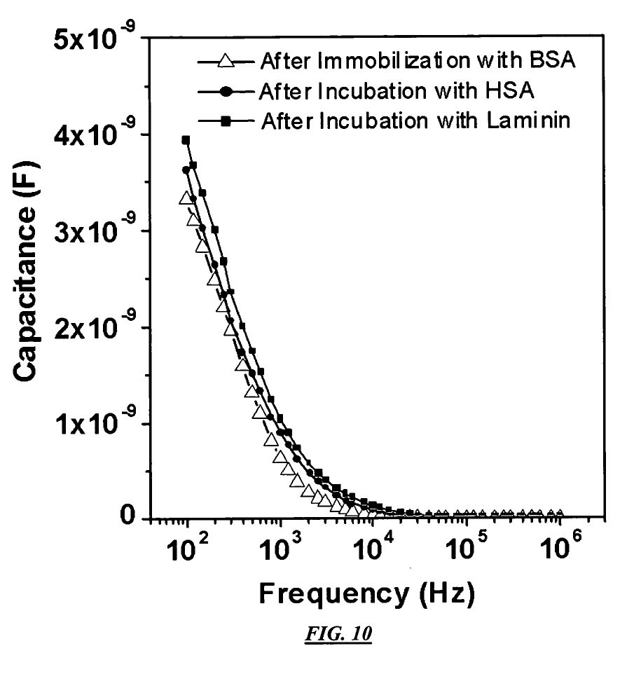

The experimental procedure started with a device with either of the two immobilization methods. Next, a negative control of 50 mg/ml HSA, which does not bind to anti-laminin, was flowed into the electrode area, allowed to incubate for 45 minutes, and washed with phosphate buffered saline (PBS) pH 7.4. Next, 50 μg/ml of laminin was allowed to incubate for 45 minutes and washed with PBS. Capacitive data from both types of immobilized devices for each experimental step can be seen in FIG. 9 and FIG. 10.

In the adsorption based immobilization protocol there was seen little selectivity for laminin binding; however, using the SAM-based immobilization technique there is achieved have high selectivity for laminin, 25% capacitance change at 100 Hz versus 4% for HSA which was 1000 times higher in concentration.

Thus, this example demonstrates selective dielectric immunosensing with high signal and selectivity by using nanometer scale gaps between electrodes which are fabricated using simple micromachining technologies. This enables arraying of many immunosensors for a point-of-care diagnostic device with multi pathogen detection capability at lower pathogen concentrations. Detection limits can vary using devices with different gap sizes.

Electrical Properties of Folding

In further embodiments, a nanogap sensor according to specific embodiments of the invention can be used to detect conformation changes of proteins. This allows a number of advantages over prior techniques, including allowing for label-free analysis; providing real-time detection of conformation changes; high sensitivity; and low frequency for large molecule.

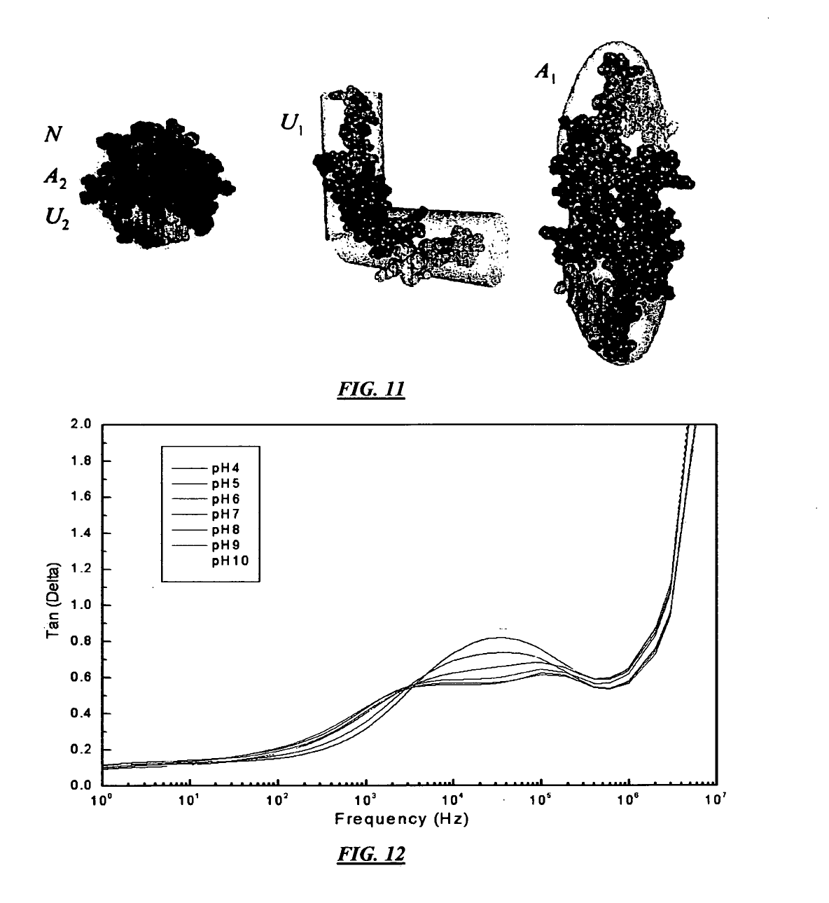

As an example, Cytochrome c is used as a sample protein to illustrate this method of operation. Cytochrome c is a well-known monomeric protein with a heme group. It can have a variety of different structures, such as the N, A2, U2: globules with different structure and radius of gyration, the U1: worm-like structure, and the A1: like dimer of globules shown in FIG. 11. These conformations can be made to vary predictably with solution pH. Its native (N), unfolded (U₁ and U₂) and molten globule (intermediate ) forms and some known transitions based on pH and salt content can be represented as follows:

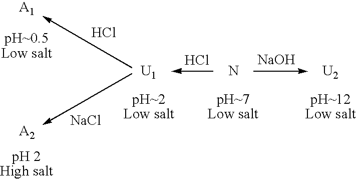

According to specific embodiments, the invention can be used to determine these different known conformations for Cytochrome c using the radius of gyration of the different forms. This technique can also be used to determine whether a polypeptide chain folds under particular conditions and to indicate kinetics of folding.

Experimental Setup

To test the ability of the nanogap to determine protein conformations, sample and control solutions were prepared and tested using an experimental setup such as that shown in FIG. 2. Control solutions of DI water were prepared with pH values: 4, 5, 6, 7, 8, 9, 10 and ionic strength: 1×10-4 M. Sample solutions were prepared at the same pH and ionic strength with concentration of Cytochrome c at 1 μM. Using this system, the pH of the Cytochrome c solutions indicates the expected radius of the Cytochrome c conformations.

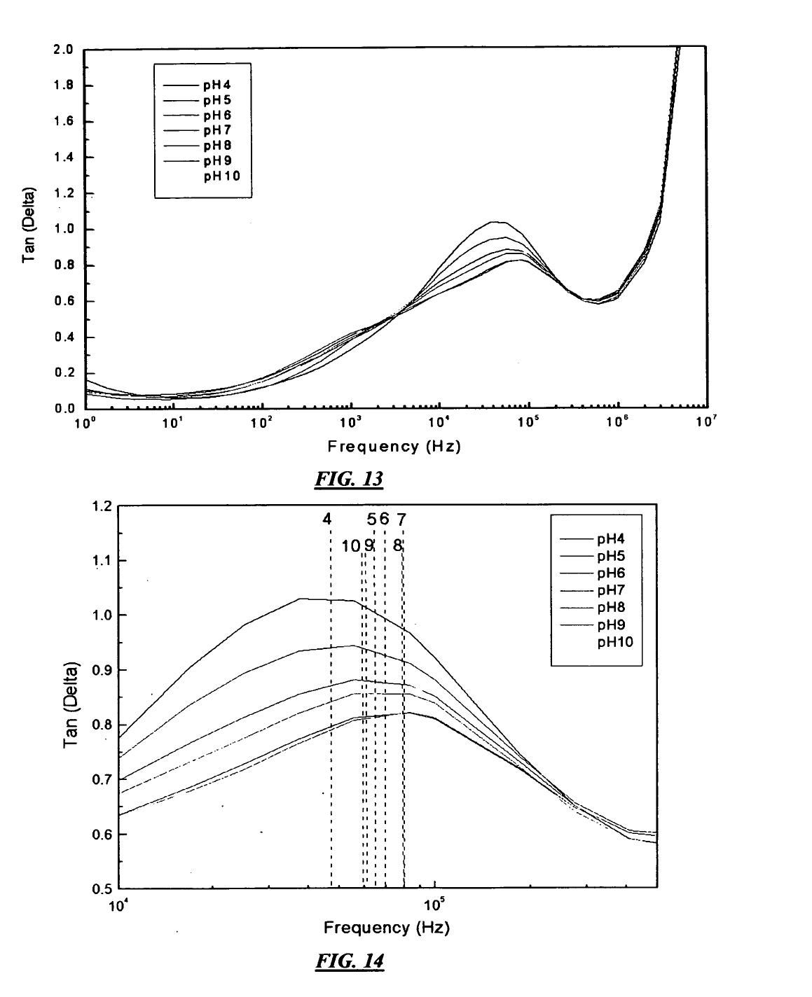

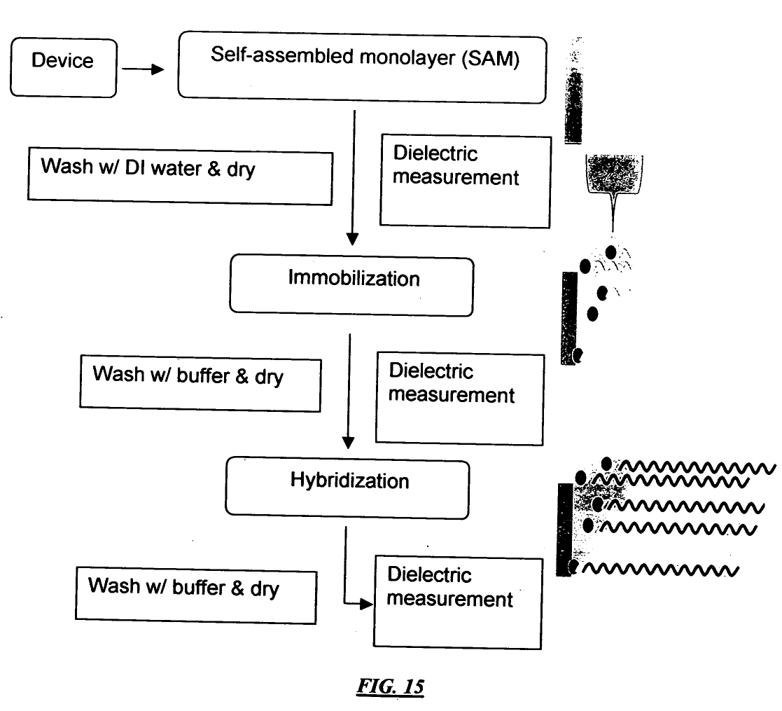

The frequency versus dielectric measurement of these concentrations is shown in FIG. 12, FIG. 13 and FIG. 14. Tan (delta) measures the Dissipation Factor (DF), which is the ratio between the permittivity and conductivity of a dielectric. The reciprocal of the DF is the quality factor, sometimes called the storage factor. In practical terms, the DF can be expressed as the ratio of the loss current to the charging current, or loss tangent.

From this data, in particular that shown in FIG. 14, the maximum Tan(delta) and Frequency at which it is occurs can be derived. This frequency can then be used to calculate a hydrodynamic radius using the formula:

$${f}^{*} = \frac{KT}{8 {π}^{2} η {r}^{3}} ⇒ r = \sqrt[3]{\frac{KT}{8 {π}^{2} η {f}^{*}}}$$

Where r: Hydrodynamic radius
K: Bolzmann' constant 1.38×10⁻²³ JK⁻¹;
T: Temperature, 300 K
η: Viscosity, 1.×10⁻³ kg/(m·s)
f*: Relaxation frequency

The Hydrodynamic Radius is the radius of a particle or polymer molecule in solution that is determined from a measurement of mobility or diffusion, for example in viscosity or dynamic light scattering experiments.

The following table summarizes the maximum relaxation frequency and calculated radius.

| pH | 4 | 5 | 6 | 7 | 8 | 9 | 10 |
|---|---|---|---|---|---|---|---|
| F | 4.80E4 | 6.30E4 | 7.00E4 | 8.05E4 | 8.00E4 | 6.05E4 | 5.95E4 |
| (Hz) | | | | | | | |
| r | 10.3 | 9.41 | 9.08 | 8.67 | 8.69 | 9.54 | 9.59 |
| (nm) | | | | | | | |

These figures have been found to highly correlate with the hydrodynamic radius determined for Cytochrome c by other methods (e.g., see Bonincontro et al., Spectrochimica Acta Part A, 59:2677-2684.)

Thus, a nanogap sensor can detect out the conformation changes of proteins in different pH solution with same ionic strength. These results demonstrate that a nanogap sensor according to specific embodiments of the invention can be used for studying protein conformations, especially for protein folding/unfolding real-time detection.

### 6. Device Fabrication

The previous methods of analysis can be accomplished use a variety of different nanogap configurations. In further embodiments, the invention is involved with novel methods for fabricating nanogap devices. While these examples involve primarily convention solid-state fabrication steps, other processes, including processing having printing, molecular growth and/or other fabrication steps as understood in the art can also be used to fabricate a device embodying the invention.

Vertical Gap

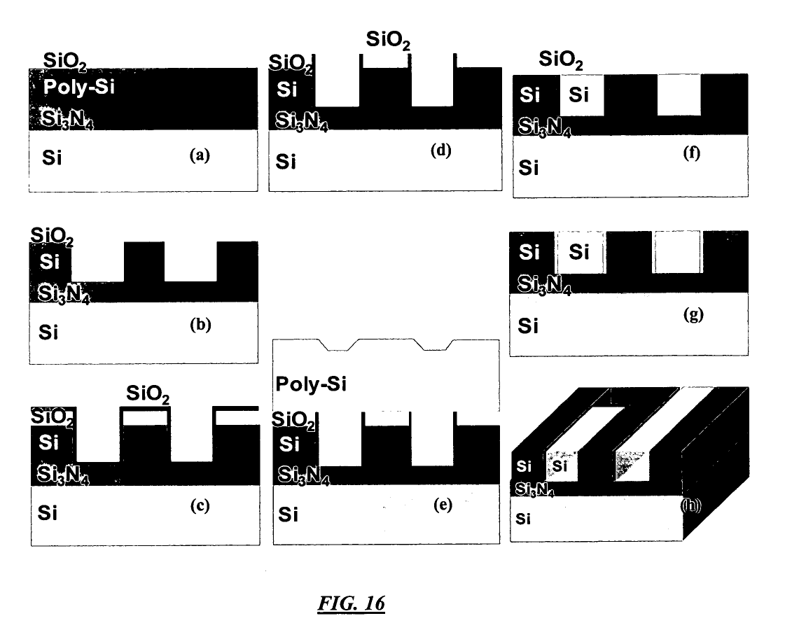

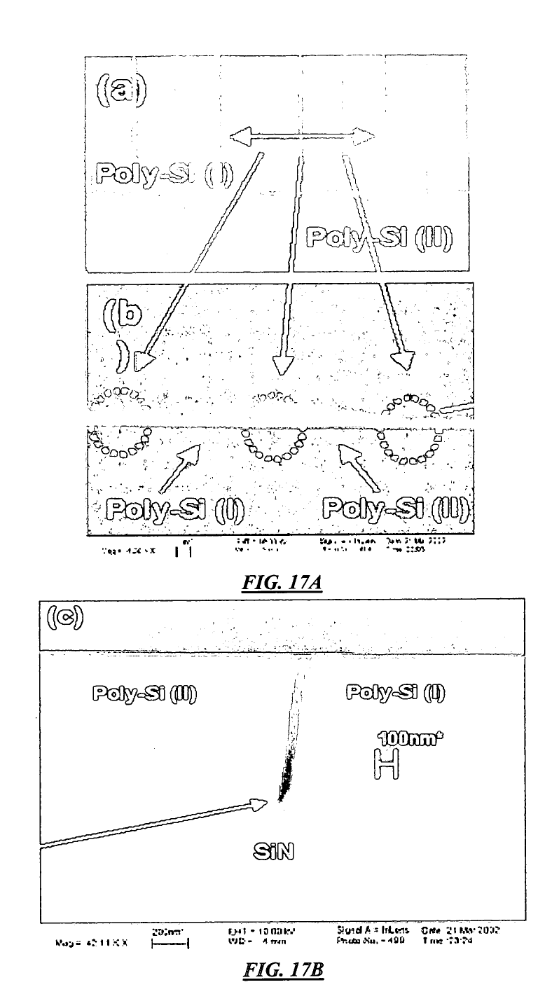

One example configuration and fabrication method is referred to herein as a “vertical gap” nanogap capicator, because the capacitor gap is configured perpendicular to the substrate on which the device is fabricated. An example embodiment was fabricated as a many-sensored array using fabrication steps that will be familiar in the art. FIG. 16 illustrates an example method for constructing a nanogap device according to specific embodiments of the present invention.

Planar

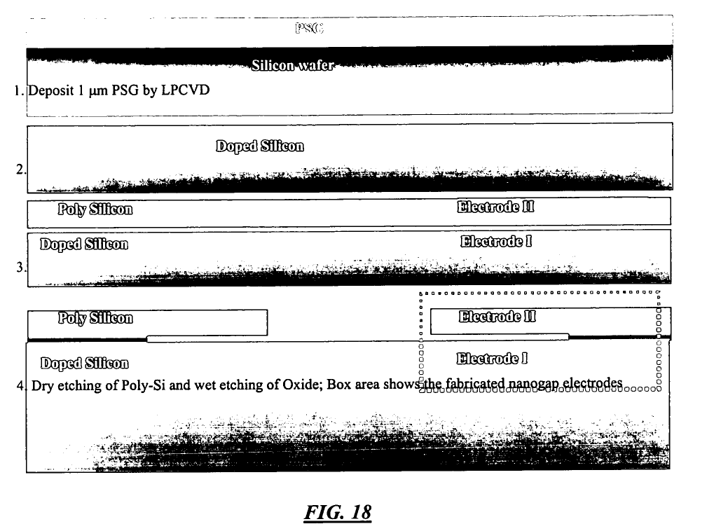

An example device is fabricated in according to specific fabrication embodiments of the present invention as illustrated in FIG. 18. In this example, a phosphosilicate glass (PSG) layer is deposited on, for example, an N-type one-sided polished silicon wafer. In this example, it provides further phosphate doping to the top of silicon wafer via an annealing process to create the bottom electrode (referred to as electrode I).

After removing the PSG, a thermal oxide film is grown. The final nanogap electrode spacing is determined by the thickness of this thermal oxide. In various specific designs, three different thicknesses of thermal oxide, e.g., approximately 20 nm, 40 nm and 100 nm can be used to provide different nanogap spaces between electrodes.

Third, an approximately 200 nanometer polysilicon layer, which acts as the top electrode (electrode II), is deposited by low pressure chemical vapor deposition (LPCVD) and plasma etched to open the door to thermal oxide. Finally, the thermal oxide is timed etched by 10:1 buffered HF solution to form the electrode gaps.

In this example device, the measured current between the two electrodes is on the order of femtoamperes at 1V after fabrication which assures that two electrodes are isolated. Before and after timed etching of the oxide layer, capacitance was measured and the amount of oxide etched was extracted. One micrometer of oxide, which defined the electrode width, was timed etched and confirmed by capacitance measurements.

This nanogap electrode by spacer nanofabrication is effective for batch processing method since it is compatible to existing silicon IC technology. This example microfabrication only needs one optical mask step and it does not need focus ion beam or e-beam-based nanolithography which are expensive and time consuming.

Planar with Molecular Sacrificial Spacer

In an alternative embodiment, a conducting material to which an SAM sacrifical layer can be attached is used to fabricate the nanogap electrodes. Gold is of particular interest in biologic type assays because it is highly inert, compared, for example to PolySi-based nanogap Gold has also be researched and used extensively as a substrate for SAM (self-assembling monolayer) applications and other chemical attachment applications. Therefore, many techniques are known for easily attaching functionalized group to gold. According to specific embodiments of the invention, SAM or SAM-type structures can be used as a sacrificial layer, in particular in gold nanogap devices. SAM sacrificial layers can control gap width down to even a few Angstroms, e.g., down to few carbon atom radii lengths.

While gold is discussed as the electrode material for the remainder of this example, it should be understood that other materials, including other metals and group IV element (e.g. silicon, germanium, etc.) if appropriately doped, can be used to form the electrode. In various materials, SAM molecules can be are readily coupled to the surface if provided with appropriate linker groups, such as a thiol group or an alcohol.

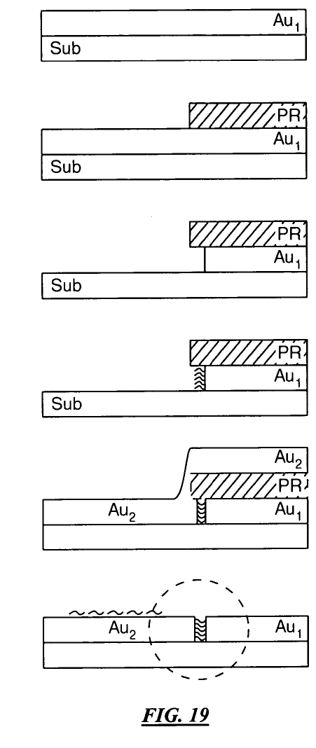

FIG. 19 is a block diagram illustrating a six step fabrication process for fabricating a nanogap device using gold (Au) deposition and to form gold electrodes with a nanogap channel according to specific embodiments of the invention. This method is illustrated from the top down. In the first step, a support substrate has a gold layer placed over it. The substrate can be any material that provides suitable mechanical and chemical support for the gold electrodes. The gold layer can be deposited using any appropriate deposition technique, including vapor, thin-film, spun, etc.

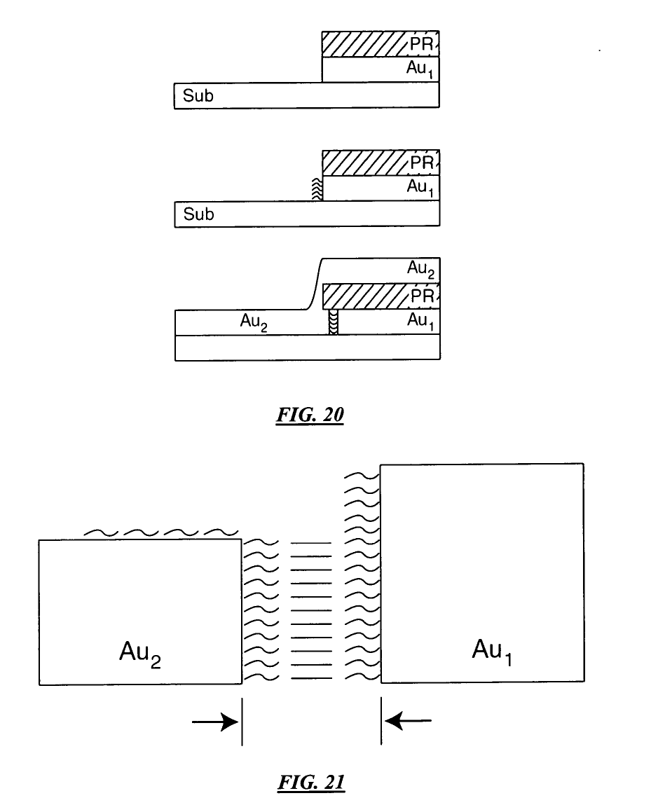

Photoresist is patterned on top of the gold layer, using any photoresist deposition and/or patterning technique. Next, the gold is etched except where protected by the photoresist. Depending on the etching, the gold layer may be underetched beneath the photoresist, as shown in FIG. 19 or etched in plane with the patterned photoresist, as shown in FIG. 20.

In the next stage, an SAM sacrifical layer is attached to the exposed portion of the first gold electrode. Thus, for example, where the exposed electrode surface is a gold surface, molecules bearing thiol groups or bearing linkers having thiol groups will self-assemble on the gold surface. The thickness of the SAM layer can be adjusted, and will determine the width of the nanogap junction.

A second gold layer is then deposited over the photoresist. This layer is deposited so that it forms a second electrode with the first electrode, with a the SAM sacrificial layer separating the two electrodes.

Finally, the photoresist and the SAM layer is removed, and the nanogap junction is is ready to receive the molecules to be dielectrically analyzed.

FIG. 21 is a block diagram illustrating a gold electrode nanogap junction with detector molecules arranged therein according to specific embodiments of the invention. In this figure, using known SAM techniques, molecules are added that form SAM layers on the gold electrode surfaces and contain or provide binding sites for molecules to be analyzed in the nanogap.

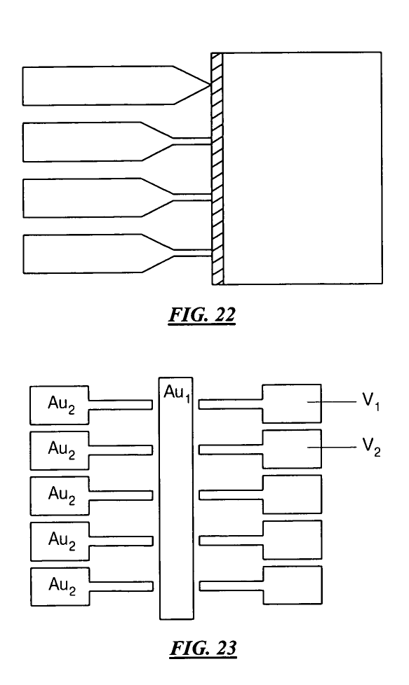

FIG. 22 is a block diagram illustrating a top view showing four first electrodes to the left, each forming a nanogap junction with an electrode to the right according to specific embodiments of the invention. FIG. 23 is a block diagram illustrating a top view showing ten first electrodes, each forming a nanogap junction with a central gold reference electrode according to specific embodiments of the invention.

Planar Nanogap Capacitor Arrays on Transparent Substrate

In an alternative configuration, a nanogap device can be fabricated as a transparent nanogap capacitor arrays on a quartz or other transparent substrate. This device allows in-situ optical bioassays coupled with dielectric spectroscopic studies. The integration of an optical window combined with dielectric nanocavities provides a solution for the calibration problem of the numbers of molecules in a nanocavity and allows developing advanced bio-optoelectronic devices. The correlation of the dielectric spectroscopy data and optical characterizations (i.e. fluorescent spectroscopy, surface enhanced Raman scattering, or surface plasma resonance) can be applied in functional genomics or single molecule detection biochip.

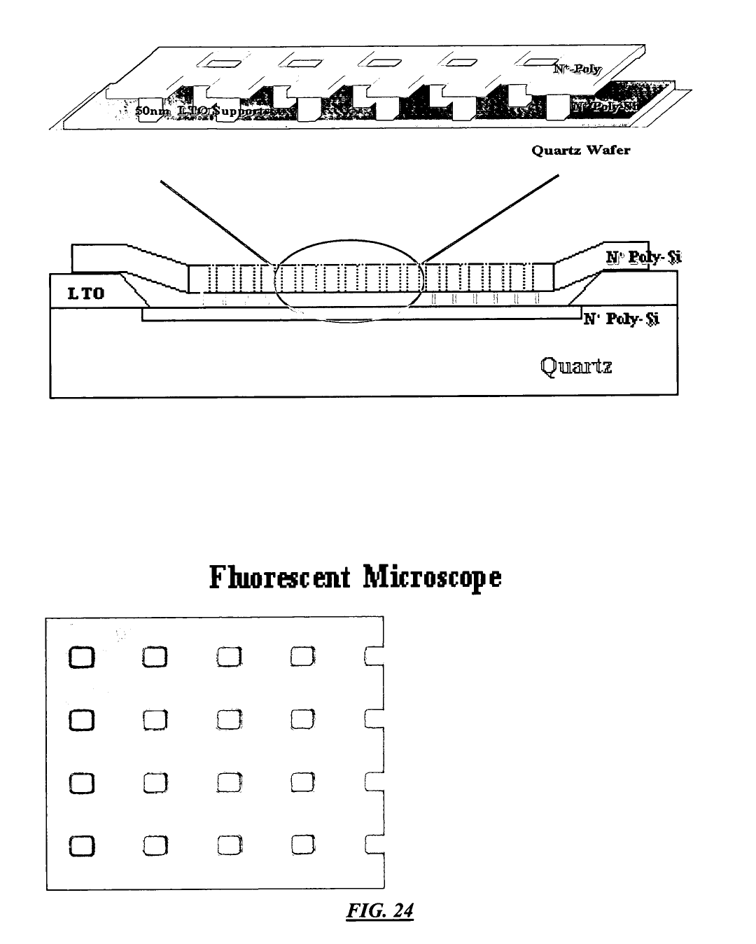

In an example embodiment, a parallel plate capacitor with 50˜100 nm gap is fabricated. FIG. 24 is a block diagram illustrating a floating or planar nano-gap capacitor with a transparent (e.g., quartz) substrate providing an optical window according to specific embodiments of the invention. The nanogap cavity allows the double layer of each electrode overlap and minimizes the potential drops.

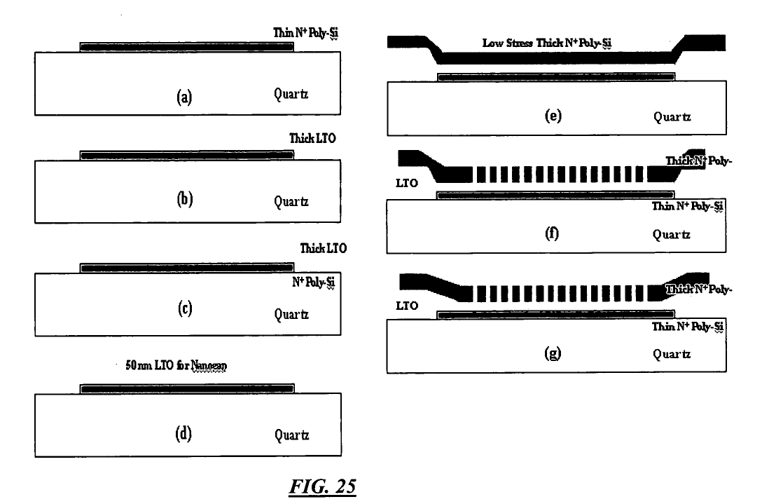

FIG. 25 is a block diagram showing steps in an example fabrication process for fabricating a transparent planar nanogap capacitor according to specific embodiments of the invention.

In a specific example, a 300×300 μm² membrane-type N⁺ poly-Si upper electrode (7000 Å) is floated by a selective in-plane etching technique and mechanically supported by intentionally controlled sacrificial oxide columns above the bottom N⁺ poly-Si electrode (500 Å) on 4-inch quartz wafers. The width of nanogap was defined by low temperature oxide (LTO) deposition of 50˜100 nm. The etch-holes of 2˜8 μm squares are differently designed to control effective electrode area. The adjacent etch-holes are separated in the range of 2˜10 μm for precise time-etch control in the N⁺ poly-Si upper electrode. In a more general example, fabrication is performed as shown in FIG. 25.

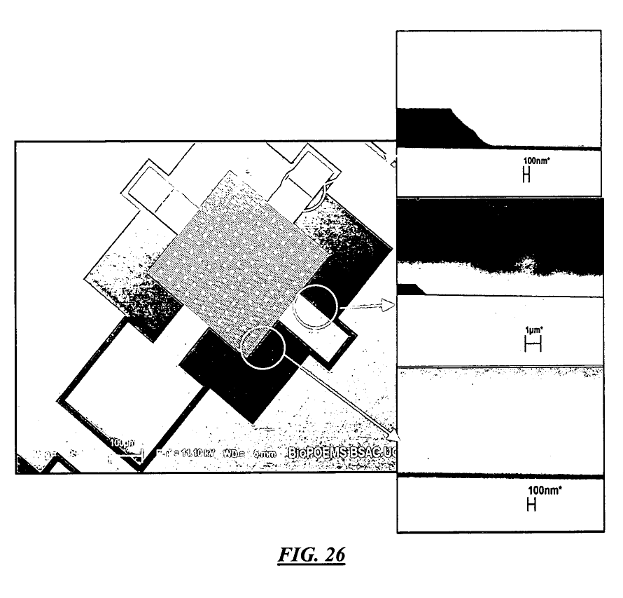

FIG. 26 shows SEM images of an example fabricated floating nanogap capacitor with 50 nm nanogap height

Optical And Dielectrical Analysis

Initial optical and dielectrical analysis for the nanogap capacitor was performed by capacitance measurement and fluorescent microscopic characterization. The permittivity of the nanogap capacitor is measured at frequencies between 100 Hz and 1 MHz, at 0V_{DC} bias and 25 mV_{AC} signals using HP4284A LCR meter. The capacitance decreases from 28 pF to 13 pF at 100 Hz after a releasing/etching process of the floating nanogap structure. Imaging using 20 nm fluorescent bead solutions of 0.2% solid concentration can be used in the nanogap capacitor to confirm the penetration of beads into the nanogap. The capacitance increases from 12 pF to 37 pF by the bead solutions.

### 7. Packaging and Experimental Setup

According to specific embodiments of the present invention, an example sensor array consists of a number of sensor elements. In a particular example array, each sensor element is addressable via mechanisms that are understood in the art and from the teachings herein. According to specific embodiments of the present invention devices, including electrical contacts and fluidic flow control can be assembled using techniques that will be understood from the art combined with the teachings of this submission and references herein.

### 8. Experimental Issues

According to specific embodiments of the present invention, an electric signal having a frequency is used to measure capacitance in a nanogap device. Dielectric dispersion issues that may relate to specific embodiments of the present invention include:
alpha dispersion: (10 Hz to a few kHz), associated with tissue interfaces such as membranes and may also be due to the relaxation of counter ions surrounding biomolecules.
beta dispersion: (1 kHz to several MHz), associated with the polarization of cellular membranes and polarization of protein and other organic macromolecules.
gamma dispersion: (greater than 10 GHz), associated with the polarization of water molecules. The interaction of water with protein is one of the significant factors that determines the properties of biopharmaceutical materials since water can cause physical and chemical instability that could ultimately lead to the loss of activity of protein. Frequencies in this range are not employed in specific embodiments.

### 9. Diagnostic Uses

As described above, following identification and validation of a detector for a particular substance, including biological molecules such as genes, proteins or any oligonucleotide or polypeptide of interest according to the invention, in specific embodiments such detectors are used in clinical or research settings, such as to predictively categorize subjects into disease-relevant classes. Detectors according to the methods the invention can be utilized for a variety of purposes by researchers, physicians, healthcare workers, hospitals, laboratories, patients, companies and other institutions. For example, the detectors can be applied to: diagnose disease; assess severity of disease; predict future occurrence of disease; predict future complications of disease; determine disease prognosis; evaluate the patient's risk; assess response to current drug therapy; assess response to current non-pharmacologic therapy; determine the most appropriate medication or treatment for the patient; and determine most appropriate additional diagnostic testing for the patient, among other clinically and epidemiologically relevant applications. Essentially any disease, condition, or status for which at least one gene is differentially expressed can be evaluated, e.g., diagnosed, monitored, etc. using the diagnostic gene sets and methods of the invention, see, e.g. Table 1.

In addition to assessing health status at an individual level, the methods and diagnostic sensors of the present invention are suitable for evaluating subjects at a “population level,” e.g., for epidemiological studies, or for population screening for a condition or disease. Expression profiles can be assessed in subject samples using the same or different techniques as those used to identify and validate the diagnostic sensors.

Web Site Embodiment

The methods of this invention can be implemented in a localized or distributed data environment. For example, in one embodiment featuring a localized computing environment, a sensor according to specific embodiments of the present invention is configured in proximity to a detector, which is, in turn, linked to a computational device equipped with user input and output features. In a distributed environment, the methods can be implemented on a single computer, a computer with multiple processes or, alternatively, on multiple computers. Sensors according to specific embodiments of the present invention can be placed onto wireless integrated circuit devices and such wireless devices can return data to a configured information processing system for receiving such devices. Such devices could, for example, be configured to be implanted in a subjects body or be released into the environment, such as for pollution or contraband detection.

Kits

A detector according to specific embodiments of the present invention is optionally provided to a user as a kit. Typically, a kit of the invention contains one or more sensors constructed according to the methods described herein. Most often, the kit contains a diagnostic sensor packaged in a suitable container. The kit typically further comprises, one or more additional reagents, e.g., substrates, tubes and/or other accessories, reagents for collecting blood samples, buffers, e.g., erythrocyte lysis buffer, leukocyte lysis buffer, hybridization chambers, cover slips, etc., as well as a software package, e.g., including the statistical methods of the invention, e.g., as described above, and a password and/or account number for accessing the compiled database. The kit optionally further comprises an instruction set or user manual detailing preferred methods of using the kit components for sensing a substance of interest.

When used according to the instructions, the kit enables the user to identify disease specific substances (such as genes and/or proteins and/or other anti-gens) using patient tissues, including, but not limited to blood. The kit can also allow the user to access a central database server that receives and provides expression information to the user. Such information facilitates the discovery of additional diagnostic gene sets by the user. Additionally, or alternatively, the kit allows the user, e.g., a health care practitioner, clinical laboratory, or researcher, to determine the probability that an individual belongs to a clinically relevant class of subjects (diagnostic or otherwise).

Embodiment in a Programmed Information Appliance

The invention may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

Integrated Systems

Integrated systems for the collection and analysis of expression profiles, molecular signatures, as well as for the compilation, storage and access of the databases of the invention, typically include a digital computer with software including an instruction set for sequence searching and/or analysis, and, optionally, one or more of high-throughput sample control software, image analysis software, data interpretation software, a robotic control armature for transferring solutions from a source to a destination (such as a detection device) operably linked to the digital computer, an input device (e.g., a computer keyboard) for entering subject data to the digital computer, or to control analysis operations or high throughput sample transfer by the robotic control armature. Optionally, the integrated system further comprises an electronic signal generator and detection scanner for probing a microarray. The scanner can interface with analysis software to provide a measurement of the presence or intensity of the hybridized and/or bound suspected ligand.

Readily available computational hardware resources using standard operating systems can be employed and modified according to the teachings provided herein, e.g., a PC (Intel x86 or Pentium chip-compatible DOS,™ OS2,™ WINDOWS,™ WINDOWS NT,™ WINDOWS95,™ WINDOWS98,™ LINUX, or even Macintosh, Sun or PCs will suffice) for use in the integrated systems of the invention. Current art in software technology is adequate to allow implementation of the methods taught herein on a computer system. Thus, in specific embodiments, the present invention can comprise a set of logic instructions (either software, or hardware encoded instructions) for performing one or more of the methods as taught herein. For example, software for providing the described data and/or statistical analysis can be constructed by one of skill using a standard programming language such as Visual Basic, Fortran, Basic, Java, or the like. Such software can also be constructed utilizing a variety of statistical programming languages, toolkits, or libraries.

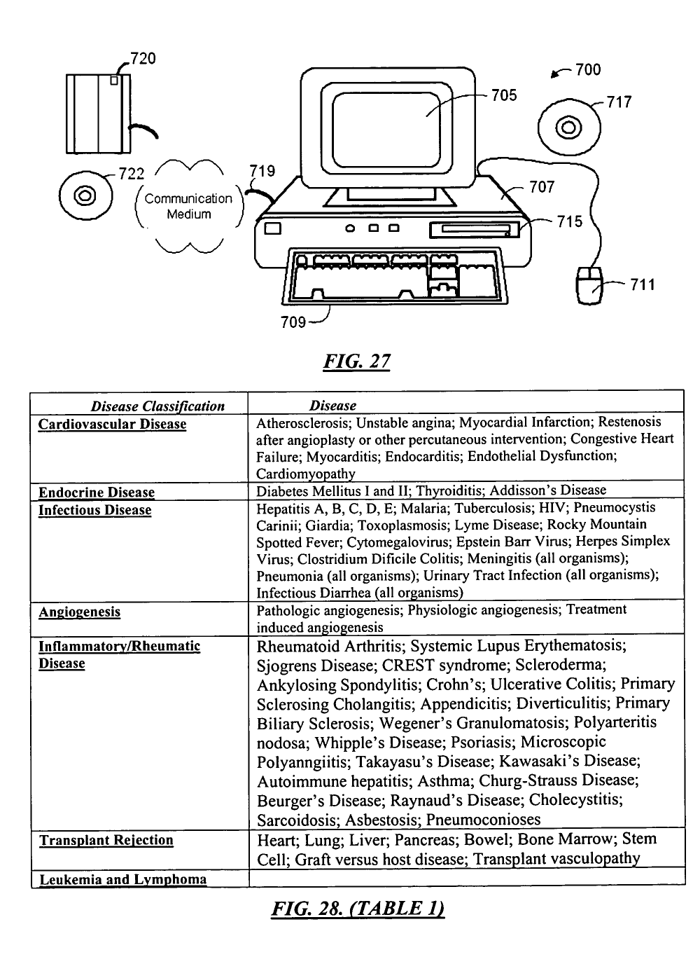

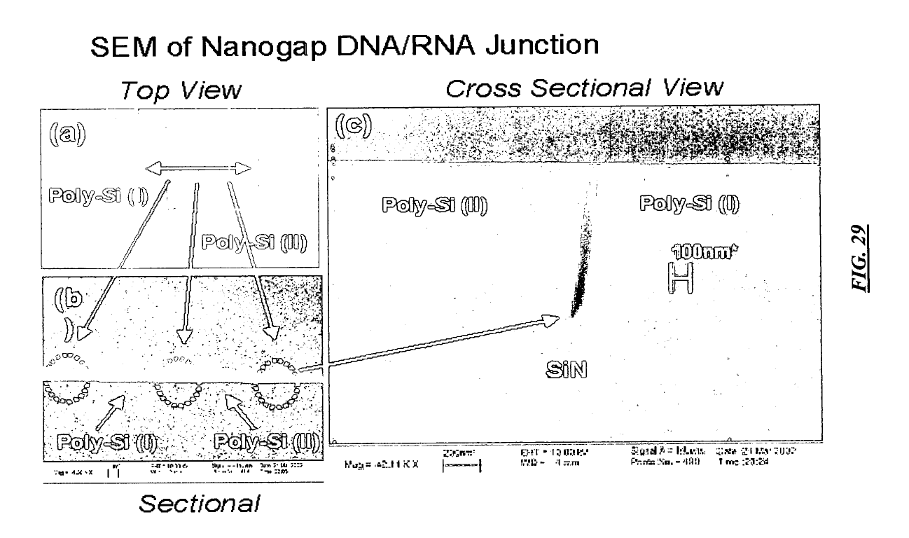

FIG. 27 is a block diagram showing a representative example logic device in which various aspects of the present invention may be embodied. FIG. 27 shows an information appliance (or digital device) 700 that may be understood as a logical apparatus that can read instructions from media 717 and/or network port 719, which can optionally be connected to server 720 having fixed media 722. Apparatus 700 can thereafter use those instructions to direct server or client logic, as understood in the art, to embody aspects of the invention. One type of logical apparatus that may embody the invention is a computer system as illustrated in 700, containing CPU 707, optional input devices 709 and 711, disk drives 715 and optional monitor 705. Fixed media 717, or fixed media 722 over port 719, may be used to program such a system and may represent a disk-type optical or magnetic media, magnetic tape, solid state dynamic or static memory, etc. In specific embodiments, the invention may be embodied in whole or in part as software recorded on this fixed media. Communication port 719 may also be used to initially receive instructions that are used to program such a system and may represent any type of communication connection.

Various programming methods and algorithms, including genetic algorithms and neural networks, can be used to perform aspects of the data collection, correlation, and storage functions, as well as other desirable functions, as described herein. In addition, digital or analog systems such as digital or analog computer systems can control a variety of other functions such as the display and/or control of input and output files. Software for performing the electrical analysis methods of the invention are also included in the computer systems of the invention.

Optionally, the integrated systems of the invention include an automated workstation. For example, such a workstation can prepare and analyze samples by performing a sequence of events including: preparing samples from a tissue or blood sample; exposing the samples to at least one array comprising all or part of a library of candidate probe molecules; and detecting the hybridization pattern by capacitance measurements. The hybridization pattern is digitized and recorded in the appropriate database.

Automated and/or semi-automated methods for solid and liquid phase high-throughput sample preparation and evaluation are available, and supported by commercially available devices. For example, robotic devices for preparation of nucleic acids from bacterial colonies, e.g., to facilitate production and characterization of the libraries of candidate genes include, for example, an automated colony picker (e.g., the Q-bot, Genetix, U.K.) capable of identifying, sampling, and inoculating up to 10,000/4 hrs different clones into 96 well microtiter dishes. Alternatively, or in addition, robotic systems for liquid handling are available from a variety of sources, e.g., automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Beckman Coulter, Inc. (Fullerton, Calif.)) which mimic the manual operations performed by a scientist. Any of the above devices are suitable for use with the present invention, e.g., for high-throughput analysis of library components or subject samples. The nature and implementation of modifications to these devices (if any) so that they can operate as discussed herein will be apparent to persons skilled in the relevant art.

Other Embodiments

Although the present invention has been described in terms of various specific embodiments, it is not intended that the invention be limited to these embodiments. Modification within the spirit of the invention will be apparent to those skilled in the art. In addition, various different actions can be used to effect a request for analysis. For example, a voice command may be spoken by the purchaser, a key may be depressed by the purchaser, a button on a client-side scientific device may be depressed by the user, or selection using any pointing device may be effected by the user.

It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this submission, including any references filed as part of an Information Disclosure Statement, are incorporated by reference in their entirety.

1. A method of detecting one or more substances of interest comprising:
exposing said one or more substances of interest to a detecting device, said device comprising a plurality of nanogaps, one or more nanogaps having one or more probe molecules attached therein and able to attach to said one or more substances of interest;
exposing said detecting device to a material suspected to contain said one or more substances of interest;
measuring a capacitance or dielectric properties at different frequency range of one or more nanogap devices; and
thereby statically and/or dynamical detecting presence of said one or more substances of interest.

2. A method of detecting conformations of one or more substances of interest comprising:
exposing said one or more substances of interest to a detecting device, said device comprising a plurality of nanogaps;
measuring a capacitance or dielectric properties at different frequency range of one or more nanogap devices; and
thereby statically and/or dynamical detecting conformations or other reaction changes of said one or more substances of interest.

3. A method of fabricating a nanogap device comprising:
placing a first selectively removable layer on a substrate surface, said substrate surface defining a horizontal orientation;
selectively removing a plurality of channels in said first layer, said channels characterized by a channel width and channel walls substantially vertical to said substrate surface;
placing a second selectively removable layer over said channels such that said second layer coats vertical sides of said channels without filling said channels, said vertical coating characterized by a vertical coating width;
placing a third layer over said layers such that said third layer fills said channels;
removing a vertical portion to expose a surface comprising regions of said first layer and regions of said third layer separated by regions of said second layer; and
removing said second layer to create a device having regions of said first layer and said third layer separated by gaps having widths largely determined by said vertical coating width.

4. The method of claim 3 further comprising:
placing a fourth selectively removable layer on said second layer prior to said selectively removing a plurality of channels.

5. The method of claim 3 further comprising:
selective removing a horizontal portion of said second layer prior to said placing said third layer.

6. The method of claim 3 further wherein:
said substrate comprises an upper layer of SiN and a lower layer of Si.

7. The method of claim 3 further wherein:
said first and/or said third layer comprises Poly-Si.

8. The method of claim 3 further wherein:
said second and/or said fourth layer comprises an oxide.

9. The method of claim 3 further wherein:
said substrate comprises any appropriate material for fabricating nanoscale devices.

10. The method of claim 3 further wherein:
said first and/or said third layer comprises any material that can be deposited on and selectively removed from said substrate.

11. The method of claim 3 further wherein:
said second layer comprises any material that can be deposited so as to provide a layer of appropriate thickness in said channels.

12. The method of claim 3 further wherein:
said channel width is a width near a smallest channel width achievable using selective mask etching.

13. The method of claim 3 further wherein:
said vertical coating width and said gap width are of similar sizes of approximately 50 nm.

14. The method of claim 3 further wherein:
said vertical coating width and said gap width are of similar sizes between approximately 5 nm and 100 nm.

15. The method of claim 3 further comprising:
attaching self-assembled monolayer (SAM) probe molecules in said gap;
exposing said gaps to material suspected to contain ligands of said probe molecules; and
detecting bindings of said ligands to said probe molecules by measuring a capacitance across said gap.

16. The method of claim 15 further wherein:
said self-assembled monolayer (SAM) probe molecules comprise single-strand oligonucleotides;
said ligands comprise one or more suspected complementary single-strand oligonucleotides;
said bindings comprise hybridization of said probe molecule and said ligands.

17. The method of claim 15 further wherein:
said self-assembled monolayer (SAM) probe molecules comprise single-strand oligonucleotides;
said ligands comprise one or more suspected complementary single-strand oligonucleotides;
said bindings comprise hybridization of said probe molecule and said ligands;
said probe molecules are in a solid state during detecting;
capacitance is measured at a range of frequency within a range of about 75 kHZ to about 5 MHZ;
two parallel electrodes used with capacitance measured between them;
said probe is a relatively short nucleotide probe of 20 mer to 40 mer.

18. A capacitor-based biodetector comprising:
a plurality of parallel electrodes arranged on a substrate with gaps between them;
a plurality of receptor probe molecules arranged between said electrodes in said gaps;
circuitry for measuring capacitance between pairs of said electrodes.

19. The device according to claim 18 further wherein:
said gaps are parallel to said substrate.

20. The device according to claim 18 further wherein:
said gaps are perpendicular to said substrate.

21. The device according to claim 18 further wherein:
said gaps are between 5 to 100 nm.

22. The device according to claim 18 further wherein:
said probe molecules comprise one or more selected from the group:
self-assembled monolayers (SAM) in said gaps;
single-strand oligonucleotides;
single-strand DNA; or
amino acid templates.

23. The device according to claim 18 further wherein:
said probe molecules comprise biologic sequence of between 20 and 60 base pairs.

24. The device according to claim 18 further wherein:
said circuitry is able to measure at a range of frequency within a range of about 25 kHZ to about 10 MHZ.

25. The device according to claim 18 further comprising:
nanoplumbing features to move substances to appropriate positions of said device.

26. A nanogap hybrid device comprising:
a plurality of gap means systematically arranged in an solid state fabricated structure;
a plurality of receptor molecules arranged in said gaps; and
means for detecting capacitance across said gaps.

27. A detector for one or more substances of interest comprising:
means for exposing said one or more substances of interest to an integrated solid state detecting device, said device having arranged therein one or more molecules able to attach to said one or more substances of interest;
means for measuring electronic characteristics of interest in small regions of said device; and
means for using measured electronic characteristics to signal the presence of said one or more substances of interest.

28. The device according to claim 26 further wherein:
said device is creating using novel nanotechnology batch-fabrication techniques;
said device comprises polysilicon chips riddled with nanogap junctions;
immobolized within each nanogap is at least one strand of reference single-strand DNA;
a voltage is applied across one or more of said nanogap junctions and a measurement is taken of capacitance;
wherein capacitance is determined by the dielectric (insulating) property of the material in the nanogap, which changes as a result of hybridization; and
wherein detecting is accomplished by adding a sample DNA and measuring a difference of capacitance after hybridization.

29. A capacitor-based biodetector device comprising:
a plurality of nanogap junctions arrays, each array comprising a plurality of nanogap junctions;
a nanofluidic network connecting to said plurality of arrays; and
a plurality of electrode connections for connecting electrical signals to said arrays.

30. The device according to claim 29 further comprising:
a plurality of receptor probe molecules arranged in said nanogap junctions.

31. The device according to claim 29 further comprising:
a covering over said plurality of arrays, said covering having at least one inlet and at least one outlet.

32. A method of fabricating a nanogap device comprising:
placing a first selectively removable electrode layer on a substrate surface, said substrate surface defining a horizontal orientation;
selectively removing a portion of said first selectively removable electrode material;
attaching a sacrificial molecular layer to a portion of said first electrode material;
placing a second selectively removable electrode layer on a substrate surface, said second electrode layer abutting said sacrificial molecular layer;
removing said sacrificial molecular layer to form a nanogap channel between said electrode layers.
